(19) United States
(12) Patent Application Publication
(10) Pub. No.: US 2021/0164012 A1
(43) Pub. Date: Jun. 3, 2021

(54) SENSOR FUNCTIONALISED BIOINK

(71) Applicants: KØBENHAVNS UNIVERSITET, Copenhagen K (DK); TECHNISCHE UNIVERSITÄT DRESDEN, Dresden (DE)

(72) Inventors: Michael KÜHL, Hornbæk (DK); Klaus KOREN, Trige (DK); Erik Christian Løvbjerg TRAMPE, Hellerup (DK); Michael GELINSKY, Meissen (DE); Anja LODE, Dresden (DE); Felix KRUJATZ, Dresden (DE); Ashwini Rahul AKKINENI, Dresden (DE)

(21) Appl. No.: 17/046,468

(22) PCT Filed: Apr. 11, 2019
(86) PCT No.: PCT/EP2019/059225

§ 371 Date: Oct. 9, 2020

(30) Foreign Application Priority Data
Apr. 11, 2018 (EP) 18166802.1

(51) Int. Cl. C12Q 1/02; C12N 5/00; C12N 1/12; C09D 11/14; B33Y 40/10; B33Y 70/00

(52) U.S. Cl. CPC C12Q 1/02; C12N 5/0068; C12N 1/12; C09D 11/14; B33Y 10/00; B33Y 70/00; C12N 2533/74; C12N 2533/78; B33Y 40/10

(57) ABSTRACT

The present invention relates to 3D printable composition comprising a cross-linkable component, a non-cross-linkable polymer, and analyte sensor particles, and to a method of fabricating a scaffold for living cells, a scaffold for a living cell, and a kit of parts comprising components for performing the method to obtain the scaffold. The scaffold is useful for prolonged culture of living cells and allows monitoring a metabolite throughout the culture with high spatial resolution of the metabolite in the scaffold.

### FIELD OF THE INVENTION

The present invention relates to a 3D printable composition, a method of fabricating a scaffold for living cells, a scaffold for a living cell, and a kit of parts comprising components for performing the method to obtain the scaffold. The scaffold is useful for prolonged culture of living cells and allows non-invasive monitoring of a metabolite or a physico-chemical parameter throughout the culture with high spatial resolution of the metabolite in the scaffold.

### PRIOR ART

The field of 3D bioprinting is making rapid progress in facilitating the construction of scaffolds containing living cells in a biocompatible matrix of various spatial complexity. Hitherto, most efforts have focused on the selection of appropriate printing matrices for particular cell types and material properties, and on monitoring cellular health and growth in the scaffolds using microscopic methods. Measurements of cellular metabolism and microenvironmental conditions have so far mostly focused on bulk measurements in media surrounding the bioprinted scaffolds, destructive sample analyses or downstream analyses of samples extracted from scaffolds.

Few examples of integrating sensors in 3D printed structures exist. Thus, WO 2016/154070 relates to tissue engineering and addresses problems relating to facilitation of transport of nutrients into engineered tissues. WO 2016/154070 discloses polymer compositions for use with 3D printers to fabricate thick, physiologically relevant vascular networks with and without cells, and the potential for oxygenating red blood cells in a prepared structure is investigated using red blood cells to illustrate the oxygenation.

Koren et al. (Environ. Sci. Technol. 49: 2286-2292, 2015) describe the incorporation of optical O₂ sensor nanoparticles into a transparent artificial sediment matrix consisting of pH-buffered deoxygenated sulfidic agar. 3D printing is not disclosed.

In light of the state of the art it remains a challenge to monitor the chemical microenvironment and metabolic activity in bioprinted cell-containing structures non-invasively. It is therefore an aim of the present invention to provide a 3D printable composition having an integrated metabolic sensor as well as a scaffold for living cells with an integrated metabolic sensor, and a method of 3D printing the scaffold.

### DISCLOSURE OF THE INVENTION

The present invention relates to a 3D printable composition comprising a cross-linkable component, a non-cross-linkable polymer, and analyte sensor particles.

The 3D printable composition is suited for fabricating, e.g. “3D printing”, a scaffold for living cells, and the analyte sensor particles allow monitoring of metabolites during culture of the living cells.

In another aspect the invention relates to a method of fabricating a scaffold for living cells. The method comprises the steps of providing a cross-linkable component, providing a non-cross-linkable polymer, providing analyte sensor particles, providing living cells, mixing the cross-linkable component, the non-cross-linkable polymer, the analyte sensor particles and the living cells in an aqueous medium to provide a bioink, and 3D printing the scaffold from the bioink. The method may also comprise the step of cross-linking the cross-linkable component. The 3D printed structure is preferably a hydrogel, and the cross-linking may strengthen the hydrogel in the 3D printed scaffold. The method preferably employs the 3D printable composition of the invention.

The present inventors have surprisingly found that when a cross-linkable component is mixed with a non-cross-linkable polymer in a 3D printable composition, the analyte sensor particles can be distributed evenly in a 3D printed scaffold without detrimental effects on the scaffold fabricated in the 3D printing, and moreover, the analyte sensor particles are firmly integrated in the scaffold while retaining their sensitivity to the analyte or metabolite. In particular, without being bound by theory the present inventors believe that the presence of the non-cross-linkable polymer prevents the analyte sensor particles from agglomerating so that the full surface area of the analyte sensor particles is available for sensing an analyte or metabolite. Thus, the method of the invention allows manufacture of a scaffold for living cells allowing non-invasive monitoring of an analyte or metabolite relevant to the living cells during culture of the living cells in the scaffold. In the context of the invention, the scaffold is suitable for living cells, and the scaffold may also be fabricated without living cells, which may be applied to the scaffold after fabrication. Thus, the bioink may contain living cells, or the bioink does not contain living cells. The even distribution of the analyte sensor particles further allows mapping, e.g. with high spatial resolution, of the analyte or metabolite in the scaffold, and thereby the cellular activity of the living cells can be monitored and mapped.

It is further contemplated within the invention that the analyte sensor particles may be replaced with sensor particles relevant for systems not involving living cells. For example, sensor particles as defined throughout this document may also be included in a fabricated, e.g. 3D printed structure, for monitoring reactants and/or products, including intermediary products, in a chemical process. Likewise, the analyte sensor particles may also monitor physical parameters, such as temperature and/or light. Sensor particles for chemical processes are not limited to scaffolds for use in aqueous environments, and the scaffold may be fabricated from any 3D printable material, e.g. polymeric materials or ceramic materials.

When the scaffold has been cross-linked after 3D printing, the analyte sensor particles, e.g. the metabolite sensor nanoparticles, e.g. in the size range of 10 nm to 500 nm, are sufficiently strongly integrated in the scaffold that no leakage of the analyte sensor particles, e.g. the metabolite sensor nanoparticles, from the 3D printed scaffold into the surrounding medium occurs. Thus, no leakage was observed over several days of incubation in a specific example. This emphasises how the scaffold of the invention is suited for prolonged culture of cells, especially sensitive cells, since the analyte sensor particles are firmly integrated in the scaffold structure. This is especially relevant for scaffolds prepared with cross-linking when the analyte sensor particles are nanoparticles.

The 3D printable composition comprises a cross-linkable component, and any cross-linkable component capable of be employed in a conventional 3D printer may be contained in the composition of the invention. The cross-linkable component will be or is capable of forming a hydrogel. In the context of the invention the cross-linkable component may also be referred to as a “cross-linkable hydrogel”. Furthermore, the hydrogel should be capable of maintaining biological cells viable. In preferred embodiments, the cross-linkable component is carbohydrate based, e.g. a carbohydrate based polymer, and exemplary carbohydrate based polymers comprise alginate, pectin, carrageenan, agar, chitosan, gellan gum, xanthan gum, gum arabic, guar gum, and locust bean gum. Other relevant polymeric cross-linkable components are polymers derived from plants, animals, bacteria or synthetic polymers and their modified versions. The cross-linkable component, e.g. the carbohydrate based polymer, may also be any mixture of these components, and the cross-linkable component may also comprise other components, e.g. organic polymers or biopolymers, excipients or the like, including cross-linkable peptides and other polymers cross-linkable by click chemistry. Click chemistry is well-known to the skilled person, and any click chemistry may be readily selected for the invention.

It is preferred that cross-linking of the cross-linkable component is performed non-covalently and that the cross-linkable component is therefore cross-linkable using non-covalent chemistry. It is however also contemplated that the cross-linkable component is selected to be cross-linkable covalently, e.g. using cross-linking molecules or photochemistry. Preferred non-covalently cross-linking polymers comprise carbohydrate polymers with acid groups, such as alginate, pectin and carrageenan; alginate, pectin, and carrageenan can be cross-linked using divalent metal ions, e.g. calcium, as a cross-linking agent. The acid groups may be carboxylate groups, sulphate groups, phosphate groups, etc. The cross-linkable component may also be a mixture of alginate, pectin, and carrageenan. Non-covalent cross-linking is generally milder to the living cells than covalent cross-linking, so that higher viability of the cells is obtained by using non-covalent cross-linking. In particular, cross-linking using divalent metal ions can be performed at ambient temperature in aqueous buffers.

The cross-linkable component may also be protein based, e.g. collagen or gelatin, and protein based cross-linkable components can likewise be cross-linked under mild conditions.

The 3D printable hydrogel composition comprises a non-cross-linkable polymer. It is to be understood that the non-cross-linkable polymer will not cross-linked under conditions employed in the 3D printing or subsequently. A preferred non-cross-linkable polymer is a carbohydrate based polymer that will not cross-link by addition of divalent cations, e.g. a cellulosic polymer. Any cellulosic polymer may be used, such as cellulose, methyl cellulose, or carboxymethylcellulose. The non-cross-linkable polymer, e.g. methyl cellulose, provides structure to the 3D printed scaffold prepared from the 3D printable composition.

The cross-linkable component is typically a polymer, e.g. a carbohydrate based polymer, and the non-cross-linkable polymer may also be a carbohydrate based polymer. The carbohydrate based polymer, i.e. both the cross-linkable and the non-cross-linkable polymer, may have any molecular size as appropriate, e.g. in the range of 10 kDa to 500 kDa, such as in the range of 50 kDa to 200 kDa. In particular, the polymer size will generally be sufficient for formation of a hydrogel, e.g. when exposed to cross-linking condition, such as by exposure to calcium ions or strontium ions.

The cross-linkable component and the non-cross-linkable polymer are preferably present in the 3D printable composition at a ratio in the range of 5:1 to 1:5 (by weight), although it is especially preferred that there is an excess of the non-cross-linkable polymer, e.g. the ratio of the cross-linkable component to the non-cross-linkable polymer is preferably in the range of 1:1 to 1:5, e.g. 1:3, by weight. When there is an excess of the non-cross-linkable polymer the 3D printable composition allows that complex scaffolds are fabricated, i.e. 3D printed, which have open structures, such as channels or the like. In particular, scaffolds can be fabricated having open structures in the micrometer range.

The 3D printable composition comprises analyte sensor particles. The analyte sensor particles may be sensitive to a chemical or physical parameter, i.e. an “analyte”. In a particular embodiment, the analyte is a metabolite, and the analyte sensor particles may also be referred to as metabolite sensor particles, and the two terms may be used interchangeably in the context of the invention. Likewise, the terms “analyte” and “metabolite” may also be used interchangeably. Thus, the analyte sensor particles may be sensitive to any analyte or metabolite as desired, and the analyte sensor particles will be capable of monitoring the concentration of the respective analyte or metabolite. Correspondingly, the analyte sensor particles may be sensitive to physical parameters. The analyte sensor particles may employ any principle for detection, and the detection may be optical, e.g. via fluorescence, luminescence or colour change, electrical, magnetic, etc. When optical detection is employed a scaffold printed from the 3D printable composition allows non-destructive, non-invasive monitoring of analytes or metabolites e.g. via imaging the optical signal, so that prolonged culture of the cells is possible with continuous monitoring of metabolites.

Optical sensor particles are well-known to the skilled person and any available optical sensor principle can readily be employed in the invention. Optical sensor particles typically include a molecule or compound that is sensitive to a specific analyte or metabolite and a reference compound or molecule that is insensitive to analytes or metabolites of relevance to a cell of interest, or the sensor particle may be sensitive to a physico-chemical parameter of interest. Thereby, the sensitive molecule or compound can detect the analyte or metabolite of interest and the signal from the sensitive molecule or compound can be correlated with the signal from the reference molecule or compound in order to quantify the analyte or metabolite of interest and also show the spatial distribution of the analyte or metabolite of interest in the scaffold, e.g. relative to the distribution of the analyte sensor particles.

In an embodiment, the analyte sensor particles employ optical detection principles and the analyte sensor particles comprise a reference molecule or compound and two or more molecules or compounds each sensitive to different analytes or metabolites. Due to the even distribution of the analyte sensor particles in the scaffold it is thereby possible to monitor the spatial distribution and concentration of two or more analytes or metabolites in the scaffold.

In an embodiment, different analyte sensor particles are included in the 3D printable composition so that different analytes or metabolites can be monitored simultaneously.

In a certain embodiment, the analyte sensor particles are sensitive to O₂. For example, the analyte sensor particles may contain Platinum(II) meso(2,3,4,5,6-pentafluoro)phenyl porphyrin (PtTFPP) as an O₂-sensitive indicator, and a coumarin reference compound, such as the dye known as Bu₃Coum. Other O₂ sensitive compounds comprise the europium based compound Eu(HPhN)₃dpp or different ruthenium, iridium or porphyrin based indicator compounds. In other embodiments, the analyte sensor particles use dyes based on boron-aza dipyrromethene (also known as aza-BODIPY) for sensing e.g. pH. Further fluorescent dyes are known to the skilled person. Preferred analyte sensor particles are capable of detecting and monitoring O₂, CO₂, H₂S, H₂O₂, pH, irradiation and temperature.

The analyte sensor particles preferably have a size in the range of 10 nm to 10 μm, and the analyte sensor particles may also be referred to as nanoparticles or microparticles. When the analyte sensor particles are nanoparticles having a size below 1 μm, e.g. with a size in the range of 10 nm to 500 nm, the large specific surface area coupled with the even distribution provided by the non-cross-linkable polymer provides a higher sensitivity to the analyte or metabolite in the scaffold than is available without the non-cross-linkable polymer.

The analyte sensor particles may be organic or inorganic as desired. For example, the analyte sensor particles may be polymeric, e.g. polystyrene or a copolymer of polystyrene, or inorganic, e.g. based on a metal oxide.

The analyte sensor particles will generally be present in an amount of 0.1% to 10% by weight of the total of the cross-linkable component and the non-cross-linkable polymer. For example, the analyte sensor particles may be provided as a suspension of 1 g/L to 10 g/L, e.g. about 5 g/L, which is mixed with the cross-linkable component and the non-cross-linkable polymer that may also be provided in a suspension.

The 3D printable composition may further comprise an aqueous medium, e.g. water or a buffered solution, such as phosphate buffered saline (PBS). The aqueous medium may also contain cellular growth factors, e.g. essential growth factors, and/or nutrients. Thus for example, the aqueous medium may be a growth medium of defined composition. When the 3D printable composition is applied to a 3D printer the concentration of the cross-linkable component and the non-cross-linkable polymer will be chosen to match the requirement of the 3D printer, although the concentration will typically be in the range of 5% to 20% by weight of the final 3D printable composition. The 3D printable composition may be provided in an aqueous medium at a higher concentration of cross-linkable component and non-cross-linkable polymer than appropriate for 3D printing so that the 3D printable composition can be diluted prior to printing. The 3D printable composition may be diluted with water, an aqueous medium, e.g. an aqueous buffer, or any other solvent considered appropriate for the specific scaffold, and the diluting medium may contain living cells of interest.

In a further aspect, the invention relates to a kit of parts comprising a cross-linkable component, a non-cross-linkable polymer, analyte sensor particles, and a cross-linking agent. The cross-linkable component, the non-cross-linkable polymer, the analyte sensor particles, and the cross-linking agent may be as defined above, and it is especially preferred that any embodiment of the 3D printable composition of the invention is included in the kit of parts. The kit of parts may also comprise an aqueous medium or other solvent, e.g. a buffered aqueous solution or a buffered solvent, or a growth medium, e.g. with growth factors and/or nutrients, and the kit of parts may contain living cells.

The method of the invention allows fabrication of a scaffold for living cells by 3D printing the bioink comprising the living cells. Any 3D printing method known to the skilled person may be employed, and any 3D printer may be used in the method of the invention. An exemplary range of 3D printers known as “BioScaffolder” is available from GeSiM—Gesellschaft für Silizium-Mikrosysteme mbH, Radeberg, Germany, or BIO-X, Holograph X, or Lumen X printers from Cellink, Gothenburg, Sweden, and 3D bioprinters from Allevi, Philadelpia, USA. 3D printers typically employ extrusion-based or ink-jet technology, e.g. piezoelectric ink-jet technology, for deposition, e.g. controlled deposition, of components. Piezoelectric inkjet technology is advantageous as it allows controlled deposition of relevant components, but photolithographic techniques for 3D bioprinting can also be employed.

When the method is performed using carbohydrate based polymers, i.e. when the cross-linkable component and the non-cross-linkable polymer are both carbohydrate based, e.g. when the cross-linkable component is alginate, pectin, carrageenan, or a mixture thereof, and when the non-cross-linkable polymer is methylcellulose, cellulose or carboxymethylcellulose, the method may be performed at ambient temperature or slightly elevated temperature, e.g. at about 37° C., so that the method is gentle to the living cells present in the bioink. For example, the method may be performed at a temperature in the range of 2° C. to 40° C. In an embodiment, the temperature is in the range of 2° C. to 6° C. In another embodiment, the temperature is in the range of 15° C. to 25° C. In a further embodiment, the temperature is in the range of 35° C. to 40° C.

In a further aspect the invention relates to a scaffold for a living cell, which scaffold comprises a cross-linked hydrogel with a non-cross-linkable polymer, and analyte sensor particles distributed in the hydrogel. The scaffold may also comprise a living cell. The scaffold is obtainable in the method of the invention and any embodiment of the 3D printable composition of the invention may be used in the fabrication of the scaffold, and likewise any embodiment of the method of the invention may be used in the fabrication of the scaffold. The scaffold allows, i.e. due to the even distribution of the analyte sensor particles and their firm integration into the structure of the scaffold, that an analyte or metabolite is monitored with high spatial resolution of the analyte or metabolite in the scaffold throughout prolonged culture of the living cell in the scaffold.

The scaffold may have any three-dimensional structure available from the 3D printer used. The scaffold advantageously has analyte sensor particles distributed in the structure, and the analyte sensor particles are firmly integrated in the scaffold, in particular when the analyte sensor particles are nanoparticles. The structure may be fabricated from a bioink comprising analyte sensor particles, but it may also comprise sections prepared using a bioink not containing analyte sensor particles. The non-cross-linkable polymer in the bioink provides that the analyte sensor particles can be distributed evenly in the structure of the scaffold and also allows that the surface area of the analyte sensor particles is fully utilised in the structure. The cross-linking of the cross-linkable component ensures that analyte sensor particles, e.g. metabolite sensor nanoparticles, are firmly integrated in the structure of the scaffold. Thus, a scaffold is obtained where living cells can be monitored non-invasively throughout a prolonged culture of the living cells. In particular, it is possible to 3D print a complex scaffold having openings, e.g. channels and the like, in the same size range as the living cells so that the living cells can be studied in an environment, e.g. with respect to shear from flowing liquids, comparable to the natural environment of the cells. For example, by using confocal microscopy it is possible to focus on selected planes in the scaffold and monitor analytes or metabolites in the plane in focus.

The living cells may be chosen freely and they may be eukaryotic, prokaryotic or archaeal cells. It is, however, preferred that the cells are eukaryotic cells. Due to the low shear available in the 3D printing it is possible to fabricate a scaffold with sensitive eukaryotic cells, e.g. mammalian cells, such as mammalian stem cells, which can subsequently be studied. It is also possible to manipulate the living cells in the scaffold, e.g. by exposing them to chemical compounds to be analysed or by controlling the physical conditions, e.g. temperature, light, electricity, radiation, magnetic fields, etc. For example, the effect of changing the conditions of the living cells can be studied.

The present invention relates to a 3D printable composition, a kit of parts comprising a 3D printable composition, a method of fabricating a scaffold for living cells, and a scaffold for a living cell. All features described for any embodiment of each aspect may be combined freely with the other aspects and any advantage mentioned for a specific aspect is readily available for other aspects by employing the corresponding feature. In particular, it is contemplated that all definitions, features, details, and embodiments for a specific aspect are available for all aspects.

### BRIEF DESCRIPTION OF THE FIGURES

In the following the invention will be explained in greater detail with the aid of an example and with reference to the schematic drawings, in which

FIG. 1 shows the viscosity of a bioink of the invention with sensor nanoparticles and of a bioink without sensor nanoparticles;

FIG. 2 shows photographs of 3D printed hydrogel scaffolds of the invention containing optical O₂ sensor nanoparticles at different O₂ levels;

FIG. 3 shows the calibration of 3D printed hydrogel scaffolds of the invention containing optical O₂ sensor nanoparticles;

FIG. 4 shows the mapping of O₂ dynamics in a 3D bioprinted scaffold containing mammalian hTERT cells and O₂ sensitive nanoparticles when exposed to a decreasing O₂ level (21-5% O₂) in the incubator atmosphere;

FIG. 5 shows the viability of the microalga Chlorella sorokiniana and the mammalian cell line hTERT in a scaffold of the invention;

FIG. 6 shows microscopic images showing the viability of the microalga C. sorokiniana (A) and the mammalian cell line hTERT (B) when immobilised in 3D bioprinted hydrogel scaffolds containing O₂ sensor nanoparticles;

FIG. 7 shows variable chlorophyll fluorescence imaging of 3D bioprinted hydrogel scaffold containing the green alga C. sorokiniana in all layers;

FIG. 8 shows the time course of O₂ concentrations in a scaffold of the invention;

FIG. 9 shows lateral profiles of O₂ concentrations in a scaffold of the invention.

Reference to the figures serves to explain the invention and should not be construed as limiting the features to the specific embodiments as depicted.

### DETAILED DESCRIPTION OF THE INVENTION

The present invention relates to 3D printable composition, a method of fabricating a scaffold for living cells, a scaffold for a living cell, and a kit of parts comprising components for performing the method to obtain the scaffold. Thus, the 3D printable composition comprises a cross-linkable component, a non-cross-linkable polymer, and analyte sensor particles.

These components are relevant also for the kit of parts, which also comprises a cross-linking agent. Further details of the aspects of the invention are disclosed above with additional details provided below.

The composition of the invention is a “3D printable” composition. In the context of the invention “3D printing” is understood in its broadest sense. 3D printing may also be referred to as “additive manufacturing” and describes fabrication of three-dimensional (“3D”) objects by additive deposition, additive agglomeration or additive layering, and also stereolithography or selective laser sintering.

In the context of the present invention a 3D printable composition may also be referred to as a “bioink”, and the two terms may be used interchangeably. The term “bioink” may be used regardless of the presence of living cells or analyte sensor particles during 3D printing and the term generally implies that a scaffold or structure fabricated from the bioink is suited for culturing living cells.

The scaffold of the invention allows non-invasive monitoring a metabolite relevant to living cells in the scaffold during culture. In the context of the invention the term “culture” is to be understood broadly, and implies that the living cells will be alive in the scaffold. The term culture does not imply that the cells are dividing, so that the living cells may also be considered to be “maintained” and the scaffold is suitable for “maintenance” of living cells.

The scaffold of the invention is suited for culture or maintenance of a living cells. In general, the term “a living cell” will refer to a plurality of living cells, although it is also contemplated that a single living cell may be included in the bioink or applied to the scaffold after fabrication.

In the context of the invention the “cross-linkable component” may be a polymer or a monomer capable of being polymerised. For polymerisable monomers any polymerisation chemistry is contemplated.

The 3D printable composition also comprises a “non-cross-linkable polymer”. Any polymer that cannot be cross-linked under conditions employed in the 3D printing process and during the optional cross-linking of the cross-linkable component is contemplated for the invention.

In the context of the present invention a structure obtainable in the method of the invention is considered to constitute a “hydrogel”. Preferred hydrogels are obtained when both the cross-linkable component and also the non-cross-linkable polymer are carbohydrate based polymers, and even more preferred hydrogels are obtained when the cross-linkable polymer, e.g. a carbohydrate based polymer with acidic groups, is cross-linked using a cross-linking agent, e.g. divalent metal ions, such as Ca²⁺.

The method may employ a cross-linking agent, and the kit of parts contains a cross-linking agent. In the context of the present invention a “cross-linking agent” is any chemical agent that can induce cross-linking of the cross-linkable component. It is, however, preferred that the cross-linking does not involve covalent reactions between the cross-linking agent and the cross-linkable component, or between two molecules of the cross-linkable component. Thus, a preferred cross-linking agent is a salt with a divalent metal ion, e.g. CaCl₂), CaBr₂, CaI₂, etc. It is however also contemplated that other divalent metals may be employed, e.g. magnesium or strontium, or transition metals. Moreover, trivalent metal ions, e.g. in the form of salts with trivalent aluminium, are also contemplated as cross-linking agents. Divalent metal ions are especially useful for cross-linking carbohydrate polymers with acidic groups.

The invention employs analyte sensor particles, and in the invention the “analyte sensor particle” uses a “detection principle” for sensing, or detecting, an analyte, in particular a metabolite. The analyte or metabolite is therefore considered to be “detectable” by the analyte sensor particle. Analyte sensor particles may also be referred to as indicator probes. In general, the detection principle is specific for a single analyte, e.g. the analyte sensor particle may be an O₂ sensor particle, although the analyte sensor particle may also be specific for additional analytes, e.g. an analyte sensor particle may also include a pH sensor. The analyte sensor particle will normally have a reference compound that improves the data, e.g. for quantification, obtained from the analyte sensor particle. However, analyte sensor particles without a reference compound are also included in the invention.

The analyte sensor particles will generally be “small”, i.e. they can be classified as “nanoparticles” or “microparticles”. In the context of the present invention a nanoparticle is a particle with a size in the range of 10 nm to 500 nm, and a microparticle is a particle with a size in the range of 0.5 μm to 10 μm. However, the analyte sensor particles may also be a mixture of nanoparticles and microparticles, e.g. the analyte sensor particles may have a size in the range of 10 nm to 10 μm. It is preferred that the analyte sensor particles have a monodisperse size distribution.

The invention will now be described in the following non-limiting example.

### Example

Preparation of O₂ Sensitive Nanoparticles

Oxygen (O₂) was selected as an appropriate metabolite, and O₂ sensitive nanoparticles were prepared. The O₂-sensitive indicator Platinum(II) meso(2,3,4,5,6-pentafluoro)phenyl porphyrin (PtTFPP) was bought from Frontier Scientific (www.frontiersci.com). A reference dye Bu₃Coum was generously provided by Dr. Sergey Borisov (Graz University of Technology). A styrene maleic anhydride copolymer (PSMA with 8% MA, Mw: 250000 g mol⁻¹) XIRAN® was provided from Polyscope (http://www.polyscope.eu). Tetrahydrofuran (THF) was obtained from Sigma-Aldrich.

The O₂-sensitive sensor nanoparticles were prepared according to Mistlberger et al. (2010). Briefly, 200 mg of PSMA, 3 mg of Bu₃Coum (reference dye) and 3 mg of PtTFPP (O₂ indicator) were dissolved in 20 g of THF. This mixture was quickly poured into 200 mL of vigorously stirred distilled water. After evaporating the THF under an air stream, the particle suspension was concentrated at elevated temperature (60° C.) until a concentration of 5 mg mL⁻¹ was reached. The particle concentration was checked by drying and subsequent weighing of 1 mL of the particle suspension. The obtained particles have a size of several hundred nm and exhibit a strongly negative zeta potential of around −30 mV as shown elsewhere (Mistlberger et al., 2010). The particles (both O₂ indicator and reference dye) can be excited by blue light (within a spectral range of 400-475 nm) and emit an O₂-dependent luminescence in the red spectral region (625-720 nm with a distinct peak at 650 nm) along with an O₂-independent fluorescence from the reference dye in the green spectral region (475-550 nm) (Mistlberger et al. 2010). The particle suspension could be stored over several weeks without any signs of sedimentation, aggregation, colour change or change in the calibration characteristics.

Preparation and Characterisation of a Hydrogel

An alginate (3%)/methylcellulose (9%) blend has previously shown to have excellent printing fidelity and good biocompatibility for printing hydrogel scaffolds of microalgae and human cell lines (Schütz et al. 2017, Lode et al. 2015) and was used in this Example.

A bioink was prepared by dissolving 30 mg mL⁻¹ alginic acid sodium salt (Sigma-Aldrich, Taufkirchen, Germany) either in distilled water in case of microalgae printing or in phosphate buffered saline (PBS) in case of mammalian cell printing followed by addition of 90 mg mL⁻¹ methylcellulose (Sigma-Aldrich; approximately MW=88 kDa). After thorough mixing, the mixture was incubated at room temperature for 1-2 h enabling swelling of the methylcellulose prior to printing. When preparing printing material containing the O₂ sensitive nanoparticles, equal amounts of a 6% (60 mg mL⁻¹) alginate solution and a 5 mg mL⁻¹ stock solution of the nanoparticles were mixed before addition of the methylcellulose, yielding a hydrogel matrix with the same final alginate concentration (3%) as in the hydrogels without nanoparticles.

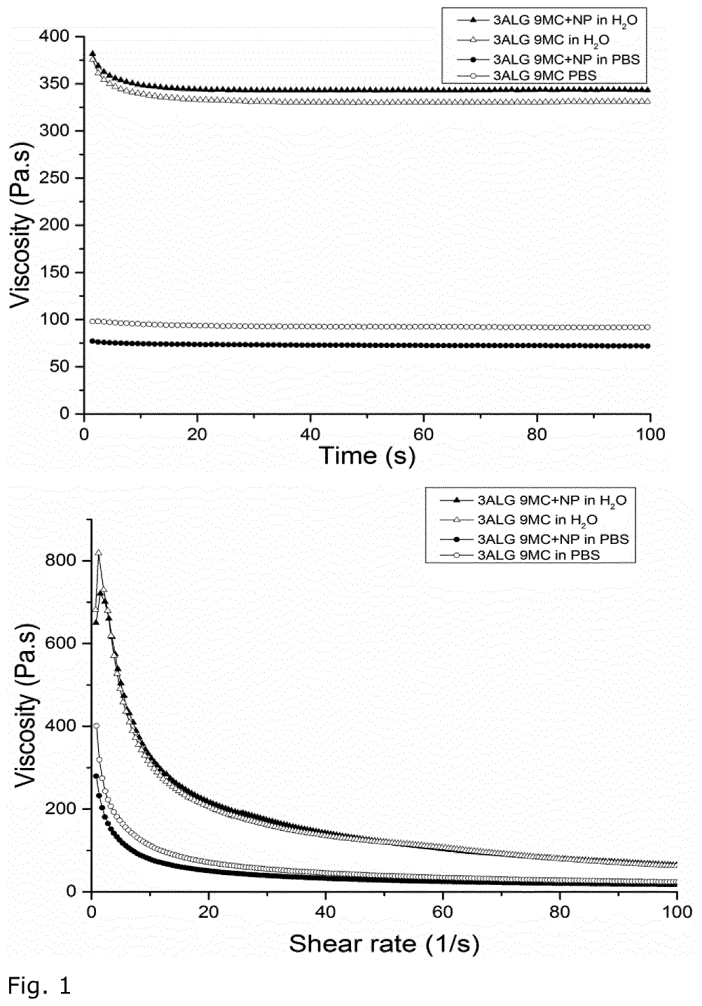

The viscosity of individual blends, e.g. 3% alginate/9% methylcellulose (in distilled water or PBS) with and without nanoparticles was measured using a rotary rheometer (Rheotest® RN 4.1, Medingen, Germany) with a 1° cone/plate. A constant shear rate of 10 s⁻¹ was applied for 100 s with a plate distance of 0.1 mm, and the corresponding viscosity was obtained for the individual blends. Shear thinning behaviour of the blends was also observed using a 1° cone/plate with a 0.1 mm plate distance. Incremental shear rate was applied over the range of 0-100 s⁻¹ (with an increment of 0.5 s⁻¹), and corresponding changes in the viscosity were quantified. A plot of shear rate and viscosity provided information about the shear thinning behaviour of the blends. See FIG. 1.

Fabrication of Hydrogel Scaffolds

Simple cross-layered, wood-pile structured hydrogel scaffolds (˜15×15×1.08 mm) were printed on a 3D plotting system (BioScaffolder 2.1, GeSiM mbH, Radeberg, Germany) as previously described (Lode et al. 2015). A 610 μm conical needle (Globaco GmbH, Rodermark, Germany) was used to print the scaffolds at a printing speed of 10 mm s⁻¹, using an extrusion pressure of ˜80 kPa and ˜300 kPa for the printing of mammalian cells and microalgae, respectively. Strand spacing was always maintained at 2 mm for all the scaffolds. An outline, e.g. a continuous strand, covering 4 sides of the scaffold was printed at an offset of 0.3 mm. The printed hydrogel scaffolds were cross-linked in a 100 mM CaCl₂) solution for 10 min before washing in PBS (for human telomerase reverse transcriptase mesenchymal stem cell scaffolds) or TAP medium (for microalgae scaffolds) and were then transferred into DMEM or TAP medium for further cultivation or calibration, respectively. While all printed scaffolds had the same overall dimensions in terms of printing geometry and thickness, scaffolds of varying composition encompassing i) pure hydrogel scaffolds, ii) scaffolds with added O₂ sensors nanoparticles, and iii) scaffolds loaded with microalgae or mammalian cells with/without added metabolite sensor nanoparticles were printed. More complex scaffolds with a pattern of crossed hydrogel layers with/without sensor nanoparticles and/or with/without cells were also printed. This included patterned mammalian/algal co-culture scaffolds (as first shown in Lode et al. 2015) of hydrogel containing O₂ sensor nanoparticles.

3D printed hydrogels containing green microalgae (Chlorella sorokiniana, strain 211-8k, obtained from the Culture Collection of Algae, University of Goettingen, Germany) or cells of an immortalised human mesenchymal stem cell line expressing hTERT (human telomerase reverse transcriptase) (Nicker et al. 2008): hTERT-MSC were prepared. Prior to printing, the microalgae were proliferated in liquid TAP (tris-acetate-phosphate) medium (50 mL in a 250 mL shaking flask) at room temperature and under a photon irradiance of 100 μmol photons m⁻² s⁻¹. The hTERT-MSC were proliferated in cell culture medium (DMEM+10% fetal calf serum+100 U/ml Penicillin and 100 μg/ml Streptomycin) at 37° C. and 5% CO₂. After harvesting, the respective alginate/methylcellulose blend was mixed with cells at a cell density of 5×10⁷ microalgal cells g⁻¹ and 5×10⁶ hTERT-MSC cells g⁻¹ of bioink, which was used for printing the cell-containing scaffolds. Similar bioink blends were used for printing scaffolds containing both cell types. After printing, hTERT-MSC scaffolds were cultivated in DMEM medium, whereas microalgal scaffolds were incubated in TP medium, i.e., TAP medium without acetic acid. Co-cultures of microalgae and hTERT-MSCs were cultivated in an adapted medium consisting of DMEM and TAP media at 37° C., 5% CO₂ and defined O₂ concentrations (1-21%) with and without illumination (white light, 450 μmol photons m⁻²s⁻¹).

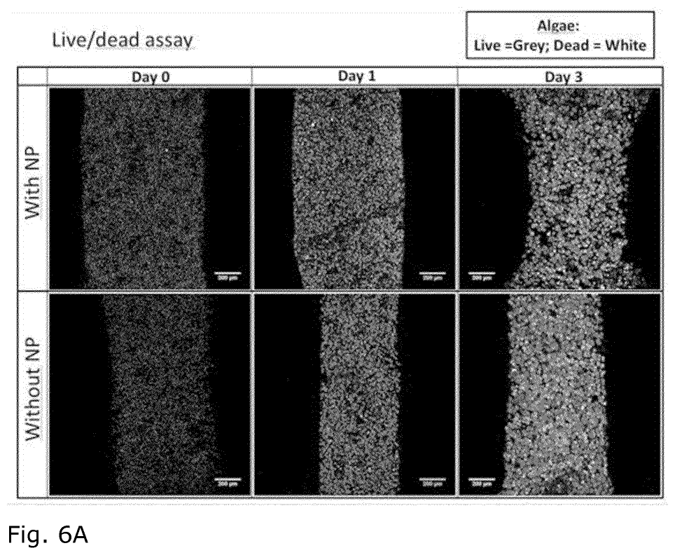

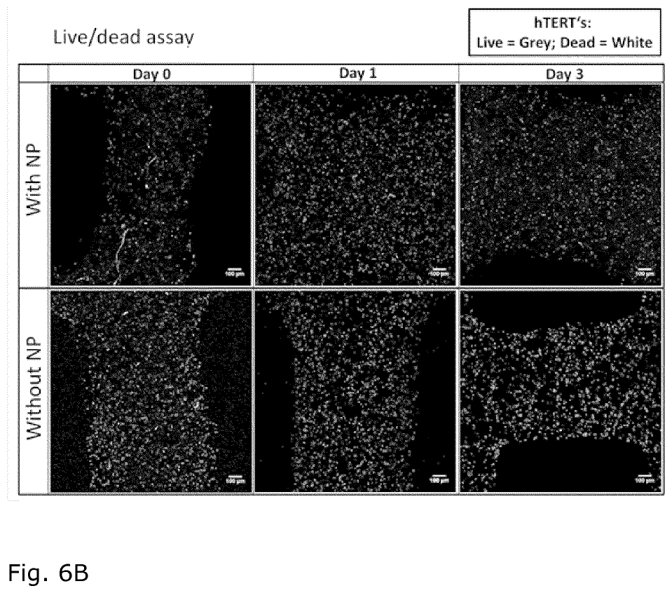

Viable cells (both hTERT-MSCs and microalgae) in printed scaffolds with nanoparticles were quantified by performing respective live-dead staining procedures immediately after printing, 24 and 72 hours after printing. Scaffolds with hTERT-MSCs and nanoparticles were incubated with 2 μM Calcein AM and 4 μM Ethidium homodimer (LIVE/DEAD Viability/Cytotoxicity Kit, for mammalian cells, Invitrogen, Eugene, Oreg., USA) for 30 min to stain live (green) and dead (red) cells. In case of scaffolds consisting of microalgae and nanoparticles, the scaffolds were incubated in TAP medium containing 5 μM SYTOX Green (dead cells stained in green) (Molecular Probes, Eugene, Oreg., USA) for 15 min at room temperature in the dark. Live cells were detected by autofluorescence of chlorophyll. After the staining procedures, z-stack images of the scaffolds were acquired using a Leica TCS SP5 confocal microscope, located at the Core Facility Cellular Imaging (CFCI) of Technische Universitat Dresden. The percentage of live and dead cells was quantified using ImageJ V1.44p (NIH; Schindelin et al 2012). See FIG. 5 and FIG. 6.

O₂ Imaging

The photosynthetic activity of the microalgae immobilised in hydrogel scaffold with O₂ sensor particles was monitored via variable chlorophyll fluorescence measurements (Schreiber 2004) using a commercial pulse-amplitude modulated imaging fluorometer (I-PAM/GFP, Walz GmbH, Effeltrich, Germany) as described in detail elsewhere (Ralph et al. 2005; Kuhl and Polerecky 2008). The system employed blue (470 nm) LED light for weak (<1 μmol photons m⁻² s⁻¹) modulated measuring light pulses, strong (0.8 s at >2500 μmol photons m⁻² s⁻¹) saturating light pulses, and defined levels of actinic irradiance, as measured with a calibrated irradiance meter at the level of bioprinted scaffolds (ULM, Walz, Effeltrich, Germany). Based on imaging the fluorescence yield under ambient light conditions, F, and under a strong light pulse completely saturating photosynthesis, F′ₘ, the effective quantum yield of photosystem (PS) II activity under a given photon irradiance (PAR) was determined as:

YII=(F′ₘ−F)/F′ₘ

Based on such YII images, a measure of the relative photosynthetic electron transport associated with PSII was calculated as rETR=PAR×YII. In absence of light, all photosynthetic reaction centres of the microalgae are open causing a minimal fluorescent yield, F₀, in darkness and a maximum fluorescent yield, Fₘ, during the brief saturation pulse. From these measurements, the maximum PSII quantum yield was calculated as:

φₘₐₓ=(Fₘ−F₀)/Fₘ

This parameter is often used as an index for fitness of photosynthetic organisms (Schreiber 2004). Besides images showing the distribution of different variable chlorophyll fluorescence-derived parameters over the 3D bioprinted hydrogel scaffolds, data could also be averaged over particular areas of interest that could be freely defined by help of the imaging systems software (ImagingWIn, Walz, Effeltrich, Germany).

A ratiometric RGB camera system was used for O₂ imaging (Larsen et al., 2011; Koren et al., 2015). The system consisted of a SLR camera (EOS 1000D, Canon, Japan) combined with a macro objective (Macro 100 f2.8 D, Tokina, Japan) equipped with a 530 nm long pass filter (Uqgoptics.com). Excitation of sensor particles was achieved with a custom-built 445 nm multichip LED equipped with a bandpass filter (NT43-156, Edmundoptics.com). The LED was powered by a USB-controlled LED driver unit for fluorescence imaging applications (http://imaging.fish-n-chips.de). Image acquisition control of the SLR and LED were done with the software look@RGB (http://imaging.fish-n-chips.de). All images of printed hydrogel scaffolds were acquired with the camera system mounted inside an incubator (HeraCell 240i Thermo Scientific, USA) kept at a constant temperature of 37° C. The incubator was capable of controlling the internal gas composition, and different defined O₂ levels could thus be adjusted. White light illumination for the scaffolds containing microalgae was provided by fiber-optic LED lamp (KL2500 LED, Schott GmbH, Germany), which could be interfaced and controlled by a PC via a USB interface, and provided defined levels of incident photon irradiance onto the scaffolds in the incubator, as measured with a calibrated photon irradiance meter (ULM, Walz GmbH, Effeltrich, Germany). All cables were guided to the outside of the incubator, which was kept closed during experiments, and all measurements were PC-controlled from the outside.

Image analysis and calibration: RGB images acquired upon LED excitation were split into red, green, and blue channels and analysed using the freeware ImageJ (http://rsbweb.nih.gov/ij/) (Koren et al. 2015). In order to obtain O₂ concentration images the following steps were performed: First the red channel (O₂ sensitive emission of the PtTFPP) and green channel (constant emission of the reference dye) images were divided using the ImageJ plugin Ratio Plus (http://rsb.info.nih.gov/ij/plugins/ratio-plus.html) in order to get the ratio image (R), which is a proxy for the O₂ dependent luminescence. In order to get O₂ concentration images from the measured ratios, a calibration curve was measured for a range of different preset O₂ levels, which was then used to convert ratio images into calibrated O₂ concentration images. Calibration was done by printing a scaffold containing only nanosensors in the bioink. The scaffold was placed in a water-filled dish inside the incubator and the O₂ levels in the incubator were changed (for zero O₂, sodium sulphite was added to the sample). A calibrated optical O₂ sensor (OXROB3 probe connected to a Firesting GO2 meter; both from PyroScience GmbH, Aachen, Germany) was also put into the incubator (in a similar volume of water) to ensure that the O₂ levels were correct and enough time was given to achieve full change of O₂. Calibration images were recorded after equilibration at each set O₂ concentration (for >2 hours). Using the curve fitting function of ImageJ all ratio images were then converted to O₂ images.

Calibration curves of optical O₂ sensors immobilised in a polymer matrix describe the collisional quenching of the indicator luminescence by O₂. The measured luminescence intensity (I) or decay time (τ) shows a non-linear decrease with O₂ concentration, [O₂], that can be described by a modified Stern-Volmer relation (Bacon and Demas 1987, Kühl 2005):

$$\frac{I}{{I}_{0}} = \frac{τ}{{τ}_{0}} = \frac{R}{{R}_{0}} = \frac{1 - α}{1 + {K}_{S V} \cdot [ {O}_{2} ]} + α \qquad Eq . 1$$

where, I₀ and τ₀ are the luminescence intensity and decay time, respectively, of the indicator in the absence of O₂, Kₛᵥ is a characteristic quenching coefficient of the optical O₂ indicator, and a describes the non-quenchable fraction of the indicator when immobilises in a given polymer matrix. In this imaging approach, the ratio between the red and green channels was used in the acquired images, R, as a proxy for the O₂ concentration dependent luminescence.

Results

The O₂ sensitive nanoparticles dispersed well in the alginate/metylcellulose mixture and formed a homogenous orange coloured printing paste that was easy to print using similar 3D printer settings previously used for bioprinting cells in similar hydrogels (Lode et al. 2015, Krujatz et al. 2015). Compared to blends without nanoparticles, addition of nanoparticles resulted in minimal increase and decrease in viscosity of 3% alginate (in distilled water)/9% methylcellulose or 3% alginate (in PBS)/9% methylcellulose, respectively. In addition, shear thinning behaviour of the blends was not affected by addition of the nanoparticles (FIG. 1), indicating that the blends are printable.

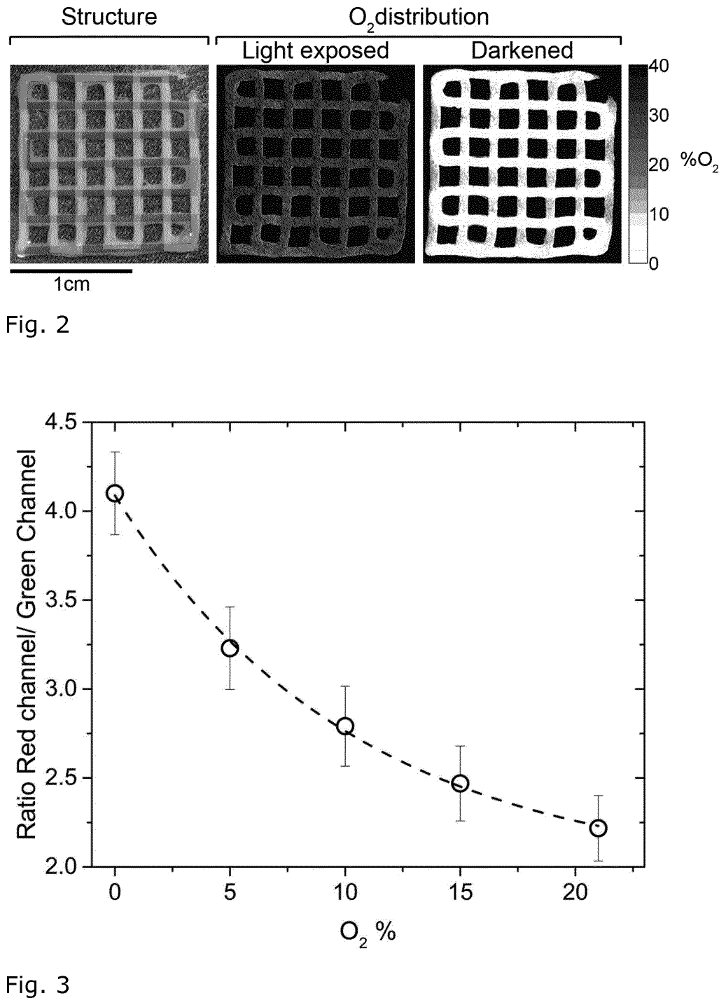

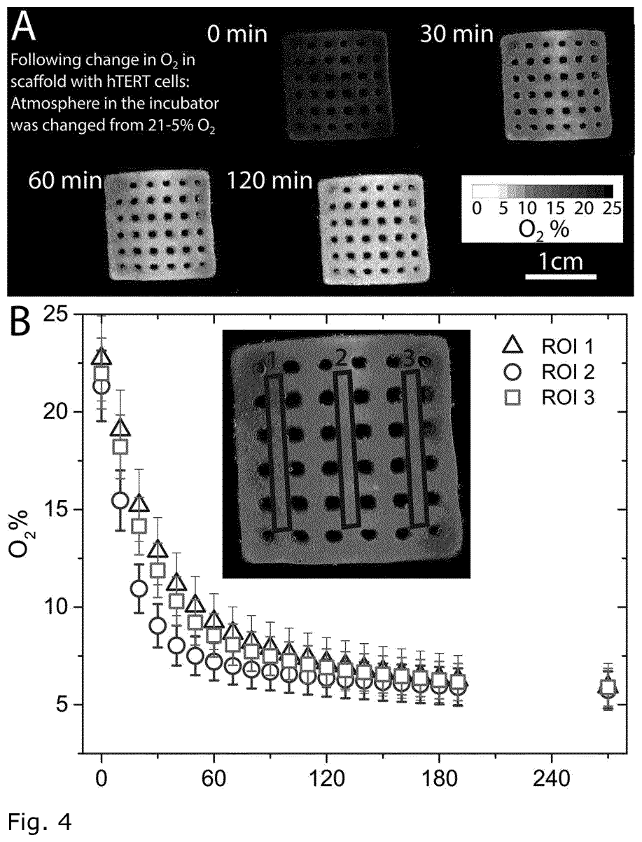

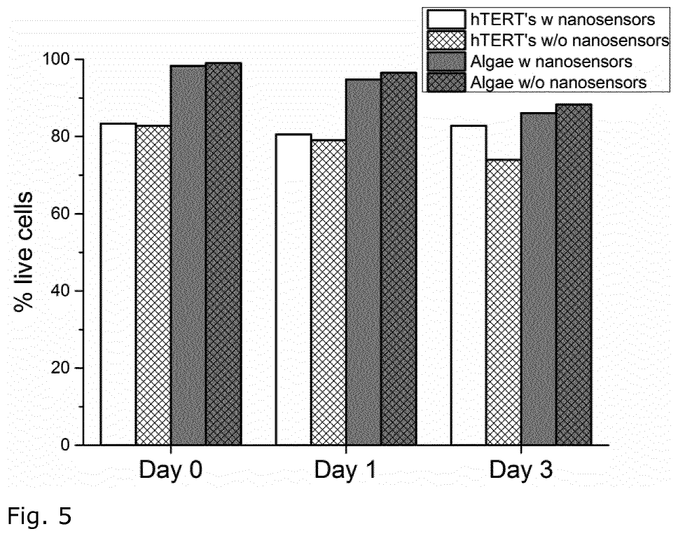

3D printed hydrogel scaffolds containing O₂ sensor particles at different O₂ levels are depicted in FIG. 2, and a calibration is shown in FIG. 3. FIG. 2 displays O₂ concentrations after a light-dark shift of a two-layered 3D bio-printed hydrogel scaffold where the vertical lanes contain microalgae and O₂ sensor particles, and the horizontal lanes contain only O₂ sensor particles; the left panel shows a photograph taken at natural daylight, and the middle and right panels show oxygen distribution in a light exposed state, and after 60 minutes and in darkness, respectively. A 3D printed hydrogel scaffolds containing only the O₂ sensitive nanoparticles showed a non-linear decrease in the ratio of O₂ dependent luminescence (detected in the red image channel) over the constant reference emission (detected in the green image channel of the camera) with increasing O₂ levels (FIG. 3) ranging from anoxia to full atmospheric saturation (21% O₂). The reaching of a new equilibrium throughout the bioprinted hydrogel scaffold after a change in external O₂ level was slow and required incubation for >4 hours for each calibration point, even with cells in the scaffold (FIG. 4). No leakage of nanoparticles from printed hydrogels scaffold into the surrounding medium was observed over several days of incubation, and we observed no significant photobleaching under the experimental irradiance levels used.

The calibration curve (FIG. 3) done at 37° C. could be fitted well by Eq. (1) (r²>0.999), yielding an apparent Kₛᵥ luminescence quenching constant of 0.077, and a non-quenchable fraction of the immobilized indicator of σ=0.258.

FIG. 5 compares the viability of the microalga C. sorokiniana and the mammalian cell line hTERT when immobilised in 3D bioprinted hydrogel scaffolds containing O₂ sensor nanoparticles. The cells were cultured in a scaffold of the invention for a prolonged period of time, and results for the first three days are shown. As judged from the proportion of live versus dead cells in the bioprinted hydrogels (FIG. 5, FIG. 6), the presence of the O₂ sensitive nanoparticles in the bioprinted hydrogel scaffolds did not reduce the viability of the immobilised microalgae and mammalian cells even after several days of incubation. The general viability of the hTERT cells was marginally lower than the microalgal cells but remained more or less constant over 3 days, while the microalgae exhibited a high but slowly decreasing viability that was, however, still >85% after 3 days (FIG. 5).

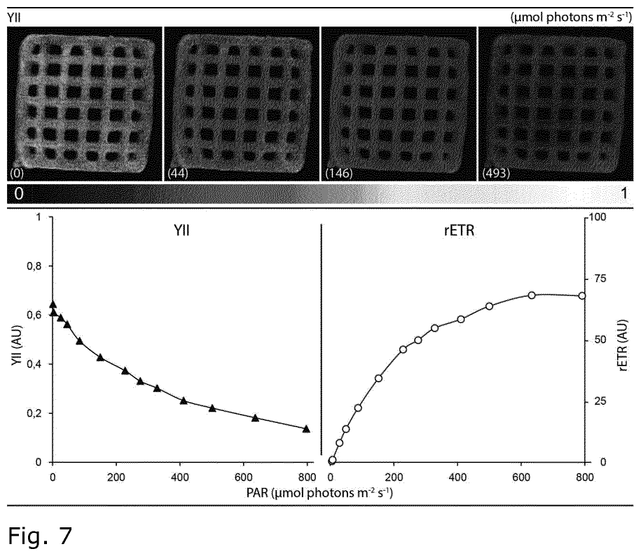

Variable chlorophyll fluorescence imaging of 3D bioprinted hydrogel scaffold containing the green alga C. sorokiniana in all layers is shown in FIG. 7. The top panel shows images of the effective quantum yield of photosystem (PS) II activity, YII, measured at increasing incident irradiance levels of blue actinic light (numbers in panels denote μmol photons m⁻² s⁻¹). Values of YSII are false colour-coded according to the coloured scale bar. In darkness, the measures YII equals the maximum PSII quantum yield, Fᵥ/Fₘ. The lower panels show a plot of YII versus photon irradiance (PAR in μmol photons m⁻² s⁻¹) (left) and the derived proxy of relative photosynthetic activity, rETR=YII×PAR, as integrated over the central vertical strand in the scaffold, showing onset of photosynthesis saturation at an irradiance of ˜250 μmol photons m⁻² s⁻¹. Good viability of microalgae in the hydrogel was confirmed by the variable chlorophyll fluorescence measurements (FIG. 7) showing a uniform high maximum PSII quantum yield of >0.6 all over the bioprinted hydrogel scaffold, and a high photosynthetic PSII activity that exhibited a typical saturation behaviour of rETR with increasing irradiance, approaching saturation above ˜250 mmol photons m⁻² s⁻¹.

The chlorophyll in the microalgae can be excited by the same wavelength (445 nm) used for excitation of the O₂ sensitive nanoparticles causing chlorophyll (Chl) fluorescence emission (680-720 nm), which can be partly detected in the red channel of the camera system, where the O₂-dependent sensor luminescence is also recorded (max emission at 650 nm). Comparison of i) Chl fluorescence from a 3D bioprinted scaffold containing only microalgae with ii) O₂ sensor luminescence from a similar 3D printed scaffold with the same microalgal density as well as nanoparticles, showed low interference from Chl fluorescence amounting to an uncertainty in the O₂ concentration determination of a few %.

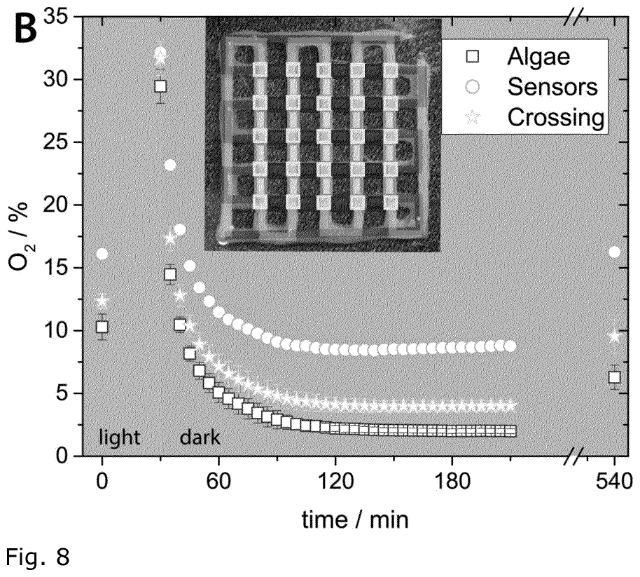

Bioprinted hydrogel scaffolds with microalgae and O₂ sensor nanoparticles demonstrated the ability of using sensor-laden bioinks in combination with luminescence imaging to map spatio-temporal chemical heterogeneity in 3D bioprinted hydrogel scaffolds. Thus, FIG. 8 shows O₂ concentrations after a light-dark shift at three different locations in a 3D printed scaffold of the invention with regions containing microalgae and O₂ sensor nanoparticles, regions with only O₂ sensor nanoparticles and regions where the two layers cross. FIG. 8 clearly shows how O₂, as a metabolite, can be monitored in distinct regions in a scaffold of the invention.

Upon incubation under high photon irradiance saturating photosynthesis (cf. FIG. 7), the overall O₂ level in the bioprinted scaffold showed supersaturating concentrations, which were then depleted during subsequent dark incubation. A steady state O₂ distribution in dark incubated scaffolds was reached about 2 hours after onset of darkness, clearly showing lowest O₂ concentrations in the hydrogel strands containing respiring microalgae. Scaffold strands with microalgae and nanoparticles encapsulated in the hydrogel exhibited the strongest O₂ concentration dynamics during experimental light-dark shifts, while O₂ changes in scaffold strands with only hydrogel and sensor nanoparticles showed less dynamics, which was mainly determined by diffusive exchange with the microalgae-laden parts of the scaffold (FIG. 8).

Local shading of the scaffold, lead to local supersaturating O₂ levels in light exposed parts of the hydrogel scaffold with microalgae, while the shaded parts exhibited lower O₂ levels, especially in the hydrogel strands with microalgae.

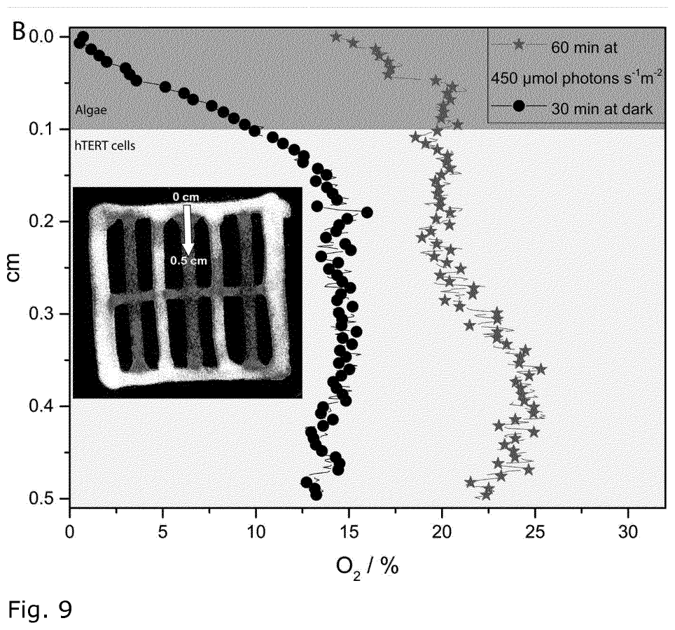

Incorporation of sensor particles in more complex 3D bioprinted hydrogel scaffolds containing hydrogel strands with microalgae, mammalian cells and pure hydrogel demonstrated the ability to map local differences in O₂ due to different metabolic activity in hydrogels compartments with mammalian and microalgal cells. For example, lateral profiles of O₂ concentration between hydrogel layer with microalgae+nanoparticles and a layer hTERT cells+sensor nanoparticles are shown in FIG. 9 (see arrow on inset) measured after 60 min light exposure and 30 min of darkness, respectively.

At contact points between sensor-laden hydrogels strands with microalgae and mammalian cells, respectively, it was possible to extract lateral O₂ concentration profiles showing how the activity of different cell types affected the O₂ availability and exchange between different compartments in the hydrogel scaffold (FIG. 9). The microalgae showed a higher respiratory activity than the mammalian cells, leading to a distinct O₂ concentration gradients, where hydrogel compartments with microalgae acted as O₂ sinks with a lower concentration than in neighbouring hydrogel compartments with mammalian cells. The O₂ concentration in hydrogel strands reached anoxia about 1 mm from intersections to hydrogel strands with mammalian cells that only depleted O₂ to about 14%. In light, the photosynthetic O₂ production alleviated the strong O₂ depletion in the hydrogel compartments with microalgae, leading to less pronounced concentration differences between compartments with mammalian cells and microalgae, where hydrogel strands with mammalian cells now became slight supersaturated by the O₂ released form the surrounding hydrogel strands with microalgae.

The above Example illustrates the utility of integrating a metabolite sensor, e.g. O₂ sensitive nanoparticles, in a bioink for 3D printing a scaffold for living cells. The O₂ sensitive nanoparticles can readily be replaced with, or supplemented with, nanoparticles capable of sensing other metabolites, e.g. CO₂, H₂S, H₂O₂, and pH, etc.

### REFERENCES

Bacon J R, and Demas J N (1987) Determination of oxygen concentrations by luminescence quenching of a polymer immobilized transition-metal complex. Anal Chem 59: 2780-2785.
Nicker W, Yin Z, Drosse I, Haasters F, Rossmann O, Wierer M, Popov C, Locher M, Mutschler W, Docheva D, and Schieker M (2008) Introducing a single-cell-derived human mesenchymal stem cell line expressing hTERT after lentiviral gene transfer. J Cell Molec Med 12: 1347-1359.
Chen M S, Zhang Y, Zhang L (2017) Fabrication and characterization of a 3D bioprinted nanoparticle-hydrogel hybrid device for biomimetic detoxification. Nanoscale 9:14506-14511.
Koren K, Brodersen K E, Jakobsen S L, and Kuhl M (2015) Optical sensor nanoparticles in artificial sediments—a new tool to visualize O₂ dynamics around the rhizome and roots of seagrasses. Environmental Science & Technology 49: 2286-2292.
Kühl M, and Polerecky L (2008) Functional and structural imaging of phototrophic microbial communities and symbioses. Aquatic Microbial Ecology 53: 99-118.
Kühl, M. (2005) Optical microsensors for analysis of microbial communities. Methods in Enzymology 397: 166-199.
Krujatz F, Lode A, Brüggemeier S, Schütz K, Kramer J, Bley T, Gelinsky M, and Weber J (2015) Green bioprinting: viability and growth analysis of microalgae immobilized in 3D-plotted hydrogels versus suspension cultures. Engineering in Life Science 15: 678-88.
Larsen M, Borisov S M, Grunwald B, Klimant I, and Glud R N (2011) A simple and inexpensive high resolution color ratiometric planar optode imaging approach: application to oxygen and pH sensing Limnology and Oceanography Methods 9: 348-360.
Liu X, Yuk H, Lin S, Parada G A, Tang T-C, Tham E, de la Fuente-Nunez C, Lu T K, and Zhao X (2018) 3D printing of living responsive materials and devices. Advanced Materials 30: 1704821.
Lode A, Krujatz F, Brüggemeier S, Quade M, Schütz K, Knaack S, Weber J, Bley T, and Gelinsky M (2015) Green bioprinting: fabrication of photosynthetic algae-laden hydrogel scaffolds for biotechnological and medical applications. Engineering in Life Science 15: 177-83.
Mistlberger G, Koren K, Scheucher E, Aigner D, Borisov S M, Zankel A, PoIt P, and Klimant I (2010) Multifunctional magnetic optical sensor particles with tunable sizes for monitoring metabolic parameters and as a basis for nanotherapeutics. Advanced Functional Materials 20: 1842-185.
Morita M, Watanabe Y, and Saiki H (2000) High photosynthetic productivity of green microalga Chlorella sorokiniana. Applied Biochemistry and Biotechnology 87: 203-218.
Ralph P J, Schreiber U, Gademann R, Kühl M, and Larkum A W D (2005) Coral photobiology studied with a new imaging PAM fluorometer. Journal of Phycology 41: 335-342.
Schindelin J et al. (2012) Fiji: an open source platform for biological image analysis. Nat Methods 9:676-682.
Schreiber U (2004) Pulse-amplitude (PAM) fluorometry and saturation pulse method. In: Papageorgiou G, Govindjee (eds) Chlorophyll fluorescence: a signature of photosynthesis. Advances in Photosynthesis and Respiration series. Kluwer Academic Publishers, Dordrecht, p 279-319.
Schütz K, Placht A-M, Paul B, Brüggemeier S, Gelinsky M, and Lode A (2017) 3D plotting of a cell-laden alginate/methylcellulose blend: towards biofabrication of tissue engineering constructs with clinically relevant dimensions. J Tissue Eng Regen Med 11: 1574-1587.
Ugwu C U, Aoyagi H, and Uchiyama H (2007) Influence of irradiance, dissolved oxygen concentration, and temperature on the growth of Chlorella sorokiniana. Photosynthetica 45: 309-311.

1. A 3D printable composition comprising
a cross-linkable component,
a non-cross-linkable polymer, and
analyte sensor particles.

2. The 3D printable composition according to claim 1, wherein the analyte detectable by the analyte sensor particles is selected from the list consisting of: O₂, CO₂, H₂S, H₂O₂, pH, irradiation and temperature.

3. The 3D printable composition according to claim 1, wherein the cross-linkable component is a carbohydrate polymer with acid groups selected from the list consisting of: alginate, pectin, and carrageenan, or a mixture thereof.

4. The 3D printable composition according to claim 1, wherein the non-cross-linkable polymer is a cellulosic polymer.

5. The 3D printable composition according to claim 1, wherein the ratio of the cross-linkable component to the non-cross-linkable polymer is in the range of 1:1 to 1:5.

6. The 3D printable composition according to claim 1, wherein the analyte sensor particles have a size in the range of 10 nm to 500 nm.

7. The 3D printable composition according to claim 1, wherein the analyte sensor particles employ optical detection principles.

8. The 3D printable composition according to claim 1 further comprising an aqueous medium.

9. The 3D printable composition according to claim 1 further comprising a living cell.

10. A method of fabricating a scaffold for living cells, the method comprising the steps of:
providing a cross-linkable component,
providing a non-cross-linkable polymer,
providing analyte sensor particles,
providing living cells,
mixing the cross-linkable component, the non-cross-linkable polymer, the analyte sensor particles and the living cells in an aqueous medium to provide a bioink,
3D printing the scaffold from the bioink.

11. The method of preparing a scaffold for living cells according to claim 10, wherein the analyte detectable by the analyte sensor particles is selected from the list consisting of: O₂, CO₂, H₂S, H₂O₂, pH, irradiation and temperature.

12. A kit of parts comprising a cross-linkable component, a non-cross-linkable polymer, analyte sensor particles, and a cross-linking agent.

13. The kit of parts according to claim 12, wherein the cross-linkable component, the non-cross-linkable polymer, and the analyte sensor particles are contained in a 3D printable composition.

14. A scaffold for a living cell, the scaffold comprising a cross-linked hydrogel with a non-cross-linkable polymer, and analyte sensor particles distributed in the hydrogel.

15. The scaffold according to claim 14 further comprising a living cell.

16. The method of preparing a scaffold for living cells according to claim 10, the method further comprising a step of cross-linking the cross-linkable component.

17. The method of preparing a scaffold for living cells according to claim 10, wherein cross-linkable component is a carbohydrate polymer with acid groups selected from the list consisting of: alginate, pectin, and carrageenan, or a mixture thereof.

18. The method of preparing a scaffold for living cells according to claim 10, wherein the non-cross-linkable polymer is a cellulosic polymer.

19. The method of preparing a scaffold for living cells according to claim 10, wherein the ratio of the cross-linkable component to the non-cross-linkable polymer is in the range of 1:1 to 1:5.

20. The method of preparing a scaffold for living cells according to claim 10, wherein the analyte sensor particles have a size in the range of 10 nm to 500 nm.

21. The method of preparing a scaffold for living cells according to claim 10, wherein the analyte sensor particles employ optical detection principles.
